(19) United States
(12) Patent Application Publication
(10) Pub. No.: US 2014/0221734 A1
(43) Pub. Date: Aug. 7, 2014

(54) CLONED NON-HUMAN ANIMALS FREE OF SELECTIVE MARKERS

(71) Applicant: Regeneron Pharmaceuticals, Inc., Tarrytown, NY (US)

(72) Inventors: Guochun Gong, Elmsford, NY (US); Ka-Man Venus Lai, Tarrytown, NY (US); David M. Valenzuela, Yorktown Heights, NY (US)

(21) Appl. No.: 14/091,207

(22) Filed: Nov. 26, 2013

Related U.S. Application Data
(60) Provisional application No. 61/730,771, filed on Nov. 28, 2012.

(51) Int. Cl. A01K 67/027; A61D 19/04; C12N 15/877

(52) U.S. Cl. CPC A01K 67/0275; C12N 15/8778; C12N 15/8771; A61D 19/04
USPC 600/34; 435/325; 435/353; 435/354

(57) ABSTRACT

Genetically modified somatic cells of a non-human animal are provided that are engineered to contain a self-excisable, recombinase expression cassette comprising a site-specific recombinase gene operably linked to an ES cell-specific promoter. Compositions and methods for producing a genetically modified, cloned non-human animal that is free of a selective marker gene and a recombinase gene are provided, wherein a targeting construct comprising a self-excisable recombinase gene operably linked to an ES cell-specific promoter is introduced into differentiated somatic cells. The genetically modified genome of the somatic cells is transferred into an enucleated host oocyte. The artificially created zygote is then cultured in vitro until the blastocyst embryonic stage and subsequently implanted into a uterus of a surrogate mother to form a genetically modified, cloned non-human animal free of selective marker and recombinase genes.

### CROSS-REFERENCE TO RELATED APPLICATION

The present application is a non-provisional of U.S. 61/730,771 filed Nov. 28, 2012, incorporated by reference in its entirety for all purposes.

### FIELD OF INVENTION

Genetically modified and cloned non-human animals that are free of a selective marker gene and a recombinase gene. Differentiated somatic cells of a non-human animal that are genetically engineered to contain a self-excisable, recombinase expression cassette comprising a site-specific recombinase gene operably linked to an ES cell-specific promoter, wherein the ES cell-specific promoter drives expression of the site-specific recombinase gene in non-differentiated pluripotent stem cells but not in differentiated somatic cells. Compositions and methods for creating a genetically modified and cloned non-human animal that is free of selective marker and site-specific recombinase genes.

### BACKGROUND OF THE INVENTION

Genetic modification techniques, e.g., transgenic, knock-in, knock-out, insertional mutagenesis, and deletion, inevitably require an insertion of a selective marker gene in the host genome in order to confirm a successful genetic modification. The selective marker gene that remains in the host genome, however, becomes unnecessary once the successful genetic modification has been confirmed and may raise safety concerns over the use of the products derived from animals containing the selective marker.

For these reasons, many efforts have been made to remove selective marker and recombinase genes from host cells or host animals following genetic modifications. For example, a recombinase gene is introduced into an ES cell or a fertilized egg, via, e.g., microinjection, transfection, or through transduction using viral particles, in order to remove a selective marker gene flanked by recombination sites, e.g., loxP or FRT. Alternatively, animals carrying a selection cassette are bred to a deleter strain that expresses a site-specific recombinase to accomplish the same effect. These techniques, however, have a number of drawbacks, including a low level of transfection efficiency in ES cells; a decrease in ES cell pluripotency due to extended in vitro culture; and requirement for additional human and financial resources for extra breeding steps.

Therefore, there is a need for compositions and methods for effectively removing selective marker and recombinase genes from genetically modified animals.

### SUMMARY OF THE INVENTION

Compositions and methods for creating genetically modified and cloned non-human animals free of a selective marker gene and a site-specific recombinase gene.

Genetically modified and cloned non-human animals, e.g., mini pigs and cows, that are free of a selective marker gene and a site-specific recombinase gene are provided, wherein the genome of the genetically modified and cloned non-human animals has been transferred from a somatic cell, e.g., fibroblast, that has been engineered to comprise a self-excisable recombinase expression cassette containing a site-specific recombinase gene operably linked to an ES cell-specific promoter. The ES cell-specific promoter drives transcription of the site-specific recombinase in undifferentiated pluripotent stem cells, e.g., in ES cells in the inner cell mass of a blastocyst-stage embryo, where ES cell-specific transcription factors are expressed and active, but not in differentiated somatic cells. Therefore, the selective marker gene and the recombinase gene, which have been introduced during genetic modification, can become removed from the genome of pluripotent stem cells during development of the cloned embryo.

Differentiated somatic cells of a non-human animal that are genetically modified to contain a self-excisable recombinase expression construct are provided, wherein the somatic cells comprise a site-specific recombinase gene operably linked to an ES cell-specific promoter, wherein the construct is flanked upstream and downstream by recombination sites oriented in the same direction with respect to each other such that the recombinase gene can be excised in the presence of the site-specific recombinase, and wherein the ES cell-specific promoter drives expression of the site-specific recombinase gene in undifferentiated pluripotent stem cells, e.g., ES cells, but not in the differentiated somatic cells. By transferring the genetically modified genome of the differentiated somatic cells into an enucleated host oocyte or into a pluripotent stem cell, where ES cell-specific transcription factors are expressed and active, the selective marker and the recombinase genes can be removed from the pluripotent stem cells in a developing cloned embryo or from any pluripotent stem cells, including somatic cells reprogrammed to be pluripotent (e.g., induced pluripotent (iPS cells)).

Methods for creating a genetically modified and cloned non-human animal that is free of a selective marker gene and a recombinase gene are provided, wherein the method comprises: (a) introducing a nucleic acid construct into differentiated somatic cells of a non-human animal to create a genetically modified genome; (b) transferring the genetically modified genome of (a) into an enucleated host oocyte; (c) fusing and activating the oocyte of (b) to form an artificial zygote; (d) culturing the artificial zygote of (c) in vitro until the zygote develops into a blastocyst embryonic stage; and (e) implanting the blastocyst of (d) into a uterus of a surrogate mother to form the genetically modified and cloned non-human animal that is free of the selective marker gene and the site-specific recombinase gene, wherein the nucleic acid construct comprises a self-excisable, recombinase expression cassette comprising a site-specific recombinase gene operably linked to an ES cell-specific promoter, wherein the recombinase expression cassette is flanked upstream and downstream by recombination sites oriented in the same direction with respect to each other such that the site-specific recombinase can be excised in the presence of the site-specific recombinase, and wherein the ES cell-specific promoter drives transcription of the site-specific recombinase gene in undifferentiated pluripotent stem cells but not in the differentiated somatic cells. Thus, once the genetically modified genome of the differentiated somatic cells is transferred into an enucleated host oocyte and the artificially created zygote is allowed to develop into an embryo, the selective marker and the recombinase genes become removed from the genome of pluripotent stem cells in a developing cloned embryo, where ES cell-specific transcription factors are expressed and active. In this way, the present invention can avoid manipulation of ES cells or any extra breeding steps required for removing selective marker and recombinase genes. In various embodiments, the nucleic acid construct is a targeting construct. In one embodiment, the targeting construct comprises a knockout allele. In one embodiment, the targeting construct comprises a knock-in allele. In one embodiment, the nucleic acid construct comprises a transgene.

Methods for producing a genetically modified and cloned pluripotent stem cell of a non-human animal that is free of a selective marker gene and a recombinase gene are provided, comprising: (a) introducing a nucleic acid construct into differentiated somatic cells of a non-human animal to create a genetically modified genome; and (b) transferring the genetically modified genome of (a) into a pluripotent stem cell to produce the genetically modified and cloned pluripotent stem cells that are free of the selective marker gene and the recombinase gene, wherein the nucleic acid construct comprises a self-excisable, recombinase expression cassette comprising a site-specific recombinase gene operably linked to an ES cell-specific promoter, wherein the recombinase expression cassette is flanked upstream and downstream by recombination sites oriented in the same direction with respect to each other such that the site-specific recombinase can be excised in the presence of the site-specific recombinase, and wherein the ES cell-specific promoter drives transcription of the site-specific recombinase gene in undifferentiated pluripotent ES cells but not in the differentiated somatic cells. In various embodiments, the nucleic acid construct is a targeting construct. In one embodiment, the targeting construct comprises a knockout allele. In one embodiment, the targeting construct comprises a knock-in allele. In one embodiment, the nucleic acid construct comprises a transgene.

The selective marker and the recombinase genes, which are flanked by recombination sites, can become removed from the genome of the pluripotent stem cells, by transferring the genetically modified genome of the differentiated somatic cells into pluripotent stem cells or any somatic cells reprogrammed to be pluripotent, where ES cell-specific transcription factors are active.

In one aspect, differentiated somatic cells of a non-human animal that are engineered to contain a self-excisable, recombinase expression cassette are provided, wherein the self-excisable, recombinase expression cassette comprises a site-specific recombinase gene operably linked to an ES cell-specific promoter, wherein the recombinase expression cassette is flanked upstream and downstream by a first and a second recombination sites that are oriented in the same direction with respect to each other such that the selective marker gene and the recombinase gene can be excised in the presence of the site-specific recombinase, and wherein the ES cell-specific promoter drives transcription of the site-specific recombinase gene in undifferentiated pluripotent stem cells but not in the differentiated somatic cells.

In one embodiment, the differentiated somatic cells are selected from the group consisting of skin cells, blood cells, nerve cells, muscle cells, bone cells, liver cells, and fat cells.

In one embodiment, the differentiated somatic cells are fibroblasts. In one embodiment, the fibroblasts are derived from a non-human animal selected from the group consisting of a mouse, a rat, a rabbit, a bird, a cow, a pig, a sheep, a goat, a horse, and a donkey. In one embodiment, the fibroblasts are derived from a pig. In a more specific embodiment, the pig is a mini pig. In one embodiment, the fibroblasts are derived from a cow.

In one embodiment, the ES cell-specific promoter is selected from the group consisting of Oct-3/4 promoter, Sox2 promoter, Kif4 promoter, c-Myc promoter, Nanog promoter, Lin28 promoter, and a combination thereof.

In one embodiment, the ES cell-specific promoter drives transcription of the site-specific recombinase gene in ES cells of a blastocyst-stage embryo.

In one embodiment, the nucleic acid construct comprises a second expression cassette between the first and the second recombination sites, wherein the second expression cassette comprises a selective marker gene operably linked to a promoter. In one embodiment, the selective marker gene is located upstream of the site-specific recombinase gene. In another embodiment, the selective marker gene is located downstream of the site-specific recombinase gene.

In one embodiment, the promoter operably linked to the selective marker gene is a constitutive promoter. In one embodiment, the constitutive promoter is selected from the group consisting of a Ubc promoter, an hCMV promoter, an mCMV promoter, an EF-1 promoter, a Pgk1 promoter, a beta-actin promoter, and a ROSA26 promoter.

In one embodiment, the selective marker is selected from the group consisting of neomycin phosphotransferase (neo^{r}), hygromycin B phosphotransferase (hyg^{r}), puromycin-Nacetyltransferase (puro^{r}), blasticidin S deaminase (bsr^{r}), xanthine/guanine phosphoribosyl transferase (gpt), and herpes simplex virus thymidine kinase (HSV-k).

In one embodiment, the self-excisable, recombinase expression construct does not comprise a selective marker gene, and the selective marker gene is located in another locus (e.g., in trans) in the genome of the differentiated somatic cells, wherein the selective marker gene is flanked upstream and downstream by third and fourth recombination sites, which are oriented in the same direction with respect to each other such that the selective marker can be removed in the presence of the site-specific recombinase. In one embodiment, the differentiated somatic cells comprise a conditional knockout allele in the genome, wherein the conditional knockout allele is flanked upstream and downstream by the first and the second recombination sites such that the conditional allele can be removed in the presence of the site-specific recombinase. In one embodiment, the conditional knockout allele further comprises a selective marker gene between the first and the second recombination sites.

In one embodiment, the nucleic acid construct comprises a nucleotide sequence homologous to at least one exon of an endogenous gene being targeted, wherein the nucleotide sequence is flanked by the first and the second recombination sites. In a more specific embodiment, the exon is a first exon of the endogenous gene.

In one embodiment, the nucleic acid construct comprises a nucleotide sequence homologous to at least one intron of an endogenous gene being targeted, wherein the nucleotide sequence is flanked by the first and the second recombination sites.

In one embodiment, the nucleic acid construct comprises a 5′-untranslated region (UTR) upstream of an initiation codon of an endogenous gene and a 3′-untranslated region (UTR) downstream of a stop codon of the endogenous gene such that the entire endogenous gene can be replaced with the nucleic acid construct via homologous recombination.

In one embodiment, the nucleic acid construct further comprises a modified sequence of an endogenous gene being targeted, wherein the modified sequence is located outside of the region flanked by the first and the second recombination sites. In one embodiment, the modified sequence is a knock-in allele of at least one exon of the endogenous gene. In one embodiment, the modified sequence is a knock-in allele of the entire endogenous gene (i.e., “gene-swap knock-in”). The knock-in allele can be an allele that confers desirable characteristics on an animal that contains the allele, such as improved disease resistance or larger size (e.g., larger muscle size). In one embodiment, the nucleic acid construct further comprises a transgene sequence, wherein the transgene sequence is located outside of the region flanked by the first and the second recombination sites. In one embodiment, the transgene sequence encodes a human protein (e.g., insulin, alpha-lactalbumin, transferrin, human serum albumin, human growth hormone, a blood clotting factor, etc.). In one embodiment, the transgene sequence encodes a therapeutic agent (e.g., a therapeutic antibody).

In one embodiment, the nucleic acid construct further comprises a modified sequence of an endogenous gene being targeted, wherein the modified sequence is a knockout allele of an endogenous gene. In one embodiment, the knockout allele comprises a reporter gene, wherein 5′ of the reporter gene comprises a nucleotide sequence immediately upstream of an initiation codon (ATG) of an endogenous gene (i.e., 5′ untranslated region (5′-UTR)) such that transcription of the reporter gene can be initiated by an endogenous promoter that drives expression of the endogenous gene, and transcription of the endogenous gene can be abolished.

In one embodiment, the reporter gene is located upstream of the first recombination site.

In one embodiment, the reporter gene encodes a reporter protein selected from the group consisting of alkaline phosphatase, luciferase, beta-galactosidase, beta-glucuronidase, green fluorescent protein (GFP), enhanced green fluorescent protein (EGFP), cyan fluorescent protein (CFP), yellow fluorescent protein (YFP), DsRed, and ZsGreen.

In one embodiment, the self-excisable, recombinase expression cassette is located in a transcriptionally active locus in the genome of the differentiated somatic cells. In one embodiment, the transcriptionally active locus is a ROSA26 locus. In one embodiment, the transcriptionally-active locus is CH25h locus.

In one embodiment, the site-specific recombinase is selected from the group consisting of Cre, Flp, and Dre recombinases.

In one embodiment, the site-specific recombinase is a Cre recombinase.

In one embodiment, the Cre recombinase comprises an intron sequence. In one embodiment, the Cre recombinase comprises a nuclear localization signal (NLS). In one embodiment, the Cre recombinase comprises both an intron sequence and a nuclear localization signal (NLS).

In one embodiment, the first and second recombination sites are selected from the group consisting of loxP, lox511, lox2272, lox66, lox71, loxM2, lox5171, FRT, FRT11, FRT71, attp, att, FRT, and Dre sites.

In one aspect, a method for producing a genetically modified and cloned non-human animal that is free of a selective marker gene and a recombinase gene is provided, the method comprising:

(a) introducing a nucleic acid construct into differentiated somatic cells of a non-human animal to create a genetically modified genome;

(b) transferring the genetically modified genome of (a) into an enucleated host oocyte;

(c) fusing and activating the oocyte of (b) to form an artificial zygote;

(d) culturing the artificial zygote of (c) until the zygote reaches a blastocyst embryonic stage; and

(e) implanting the blastocyst of (d) into a uterus of a surrogate mother to form the genetically modified and cloned non-human animal that is free of the selective marker gene and the recombinase gene,

wherein the nucleic acid construct comprises a self-excisable, recombinase expression cassette comprising a site-specific recombinase gene operably linked to an ES cell-specific promoter, wherein the recombinase expression construct is flanked upstream and downstream by a first and second recombination sites that are oriented in the same direction with respect to each other such that the site-specific recombinase can be excised in the presence of the site-specific recombinase, and wherein the ES cell-specific promoter drives transcription of the site-specific recombinase gene in undifferentiated pluripotent stem cells but not in the differentiated somatic cells. Once the modified genome of the differentiated somatic cells is transferred into an enucleated host oocyte, and the artificially created zygote is allowed to develop into a cloned embryo, where ES-cell specific transcription factors are active in pluripotent stem cells, the selective marker and the recombinase genes are removed from the genome of the cloned embryo.

In one embodiment, the nucleic acid construct is a targeting construct. In one embodiment, the targeting construct comprises a knockout allele. In one embodiment, the targeting construct comprises a knock-in allele. In one embodiment, the nucleic acid construct comprises a transgene.

In one embodiment, the ES cell-specific promoter is selected from the group consisting of Oct-3/4 promoter, Sox2 promoter, Kif4 promoter, c-Myc promoter, Nanog promoter, Lin28 promoter, and a combination thereof.

In various embodiments, the self-excisable, recombinase expression cassette comprises a second expression cassette located between the first and the second recombination sites, wherein the second expression cassette comprises a selective marker gene operably linked to a promoter. In one embodiment, the selective marker is located upstream of the site-specific recombinase gene. In another embodiment, the selective marker is located downstream of the site-specific recombinase gene.

In one embodiment, the promoter operably linked to the selective marker gene is a constitutive promoter. In one embodiment, the constitutive promoter is selected from the group consisting of a Ubc promoter, an hCMV promoter, an mCMV promoter, an EF-1 promoter, a Pgk1 promoter, a beta-actin promoter, and a ROSA26 promoter.

In one embodiment, the selective marker is selected from the group consisting of neomycin phosphotransferase (neo^{r}), hygromycin B phosphotransferase (hyg^{r}), puromycin-Nacetyltransferase (puro^{r}), blasticidin S deaminase (bsr^{r}), xanthine/guanine phosphoribosyl transferase (gpt), and herpes simplex virus thymidine kinase (HSV-k).

In one embodiment, the self-excisable, recombinase expression construct does not comprise a selective marker gene, and the selective marker gene is located in another locus (e.g., in trans) in the genome of the differentiated somatic cells, wherein the selective marker gene is flanked upstream and downstream by third and fourth recombination sites that are oriented in the same direction with respect to each other such that the selective marker can be removed in the presence of the site-specific recombinase. In one embodiment, the differentiated somatic cells comprise a conditional knockout allele in the genome, wherein the conditional knockout allele is flanked upstream and downstream by the first and the second recombination sites such that the conditional allele can be removed from the genome in the presence of the site-specific recombinase. In one embodiment, the conditional knockout allele comprises a selective marker gene between the first and the second recombination sites.

In one embodiment, the nucleic acid construct comprises a nucleotide sequence homologous to at least one exon of an endogenous gene being targeted, wherein the nucleotide sequence is flanked by the first and the second recombination sites. In one embodiment, the exon is a first exon of the endogenous gene.

In one embodiment, the nucleic acid construct comprises a nucleotide sequence homologous to at least one intron of an endogenous gene being targeted, wherein the nucleotide sequence is flanked upstream and downstream by the first and the second recombination sites.

In one embodiment, the nucleic acid construct is a targeting construct and targeting arms of the targeting construct comprise a 5′-untranslated region (UTR) upstream of an initiation codon of an endogenous gene and a 3′-untranslated region (UTR) downstream of a stop codon of the endogenous gene such that the entire endogenous gene can be replaced with the targeting construct via homologous recombination. In one embodiment, the targeting arms comprise a 5′-UTR region immediately upstream of an initiation codon of the endogenous gene. In one embodiment, targeting arms comprise a 3′ untranslated region immediately downstream of a stop codon of the endogenous gene.

In one embodiment, the nucleic acid construct further comprises a modified sequence of the endogenous gene being targeted, wherein the modified sequence is located outside of the region flanked by the first and the second recombination sites. In one embodiment, the modified sequence is a knock-in allele of at least one exon. In one embodiment, the modified sequence is a knock-in allele of the entire gene (i.e., “gene-swap knock-in”). The knock-in allele can be an allele that confers desirable characteristics on an animal that contains the allele, such as improved disease resistance or larger size (e.g., larger muscle size). In one embodiment, the nucleic acid construct further comprises a transgene sequence, wherein the transgene sequence is located outside of the region flanked by the first and the second recombination sites. In one embodiment, the transgene sequence encodes a human protein (e.g., insulin, alpha-lactalbumin, transferrin, human serum albumin, human growth hormone, a blood clotting factor, etc.). In one embodiment, the transgene sequence encodes a therapeutic agent (e.g., a therapeutic antibody).

In one embodiment, the nucleic acid construct further comprises a modified sequence of the endogenous gene being targeted, wherein the modified sequence is a knockout allele of an endogenous gene. In one embodiment, the knockout allele comprises a reporter gene, wherein 5′ of the reporter gene comprises a nucleotide sequence immediately upstream of an initiation codon (ATG) of the endogenous gene (i.e., 5′-untranslated region (5′-UTR)) such that transcription of the reporter gene is initiated by an endogenous promoter that drives the endogenous gene, and transcription of the endogenous gene is abolished.

In one embodiment, the reporter gene is located upstream of the first recombination site. In one embodiment, the reporter gene encodes a reporter protein selected from the group consisting of green fluorescent protein (GFP), enhanced green fluorescent protein (EGFP), cyan fluorescent protein (CFP), yellow fluorescent protein (YFP), DsRed, ZsGreen, and lacZ.

In one embodiment, the genetically modified genome of the differentiated somatic cells is transferred into the enucleated host oocyte via a somatic cell nuclear transfer technique (SCNT).

In one embodiment, the genetically-modified genome of the differentiated somatic cells is microinjected into a perivitelline space (i.e., the space between the zona pellucida and the cell membrane) of the enucleated host oocyte.

In one embodiment, the expression construct comprises a selective marker gene operably linked to a promoter. In one embodiment, the promoter is a constitutive promoter. In one embodiment, the constitutively active promoter is selected from the group consisting of a Ubc promoter, an hCMV promoter, an mCMV promoter, an EF-1 promoter, a Pgk1 promoter, a beta-actin promoter, and a ROSA 26 promoter.

In one embodiment, the selective marker gene is located upstream of the site-specific recombinase gene. In one embodiment, the selective marker gene is located downstream of the site-specific recombinase.

In one embodiment, the selective marker is a drug resistant gene selected from the group consisting of neomycin phosphotransferase (neo^{r}), hygromycin B phosphotransferase (hyg^{r}), puromycin-Nacetyltransferase (euro^{r}), blasticidin S deaminase (bsr^{r}), xanthine/guanine phosphoribosyl transferase (gpt), and herpes simplex virus thymidine kinase (HSV-k).

In one embodiment, the site-specific recombinase is selected from the group consisting of Cre, Flp, and Dre recombinases.

In one embodiment, the site-specific recombinase is a Cre recombinase.

In one embodiment, the Cre recombinase comprises an intron sequence. In one embodiment, the Cre recombinase comprises a nuclear localization signal (NLS). In one embodiment, the Cre recombinase comprises both an intron sequence and a nuclear localization signal (NLS).

In one embodiment, the first and second recombination sites are selected from the group consisting of loxP, lox511, lox2272, lox66, lox71, loxM2, lox5171, FRT, FRT11, FRT71, attp, att, FRT, and Dre sites.

In one aspect, a method for making a genetically modified cow or pig is provided, comprising a step of genetically modifying a somatic cell of a pig or cow to include a self-excising cassette comprising a recombinase gene driven by a promoter that is active in a pluripotent cell, and a selection gene flanked by recombinase sites to form a genetically modified pig or cow genome; and introducing the genetically modified genome into a suitable oocyte, culturing the oocyte to a blastocyst stage, gestating the blastocyst in a suitable surrogate mother, and allowing the blastocyst to develop into a genetically modified progeny.

In one aspect, a cloned oocyte of a non-human animal comprising a genetically modified genome from a differentiated somatic cell is provided, wherein the genetically modified genome comprises a nucleic acid construct containing a self-excisable, recombinase expression cassette in which a site-specific recombinase gene is operably linked to an ES cell-specific promoter, wherein the recombinase expression cassette is flanked by recombination sites oriented in the same direction with respect to each other such that the site-specific recombinase can be excised in the presence of the site-specific recombinase.

In one embodiment, the non-human animal is selected from the group consisting of a mouse, a rat, a rabbit, a bird, a cow, a pig, a sheep, a goat, a horse, and a donkey.

In one embodiment, the differentiated somatic cell is selected from the group consisting of a skin cell, a blood cell, a nerve cell, a muscle cell, a bone cell, a liver cell, and a fat cell.

In one embodiment, the differentiated somatic cell is a fibroblast. In one embodiment, the fibroblast is derived from a non-human animal selected from the group consisting of a mouse, a rat, a rabbit, a bird, a cow, a pig, a sheep, a goat, a horse, and a donkey. In one embodiment, the fibroblast is derived from a pig. In a more specific embodiment, the pig is a mini pig. In one embodiment, the fibroblast is derived from a cow.

In one embodiment, the ES cell-specific promoter is selected from the group consisting of Oct-3/4 promoter, Sox2 promoter, Kif4 promoter, c-Myc promoter, Nanog promoter, Lin28 promoter, and a combination thereof.

In one embodiment, the nucleic acid construct comprises a second expression cassette between the first and the second recombination sites, wherein the second expression cassette comprises a selective marker gene operably linked to a promoter. In one embodiment, the selective marker gene is located upstream of the site-specific recombinase gene. In another embodiment, the selective marker gene is located downstream of the site-specific recombinase gene.

In one embodiment, the promoter operably linked to the selective marker gene is a constitutive promoter. In one embodiment, the constitutive promoter is selected from the group consisting of a Ubc promoter, an hCMV promoter, an mCMV promoter, an EF-1 promoter, a Pgk1 promoter, a beta-actin promoter, and a ROSA26 promoter.

In one embodiment, the selective marker is selected from the group consisting of neomycin phosphotransferase (neo^{r}), hygromycin B phosphotransferase (hyg^{r}), puromycin-Nacetyltransferase (puro^{r}), blasticidin S deaminase (bsr^{r}), xanthine/guanine phosphoribosyl transferase (gpt), and herpes simplex virus thymidine kinase (HSV-k).

In one embodiment, the self-excisable, recombinase expression construct does not comprise a selective marker gene, and the selective marker gene is located in another locus (e.g., in trans) in the genome of the differentiated somatic cells, wherein the selective marker gene is flanked upstream and downstream by third and fourth recombination sites, which are oriented in the same direction with respect to each other such that the selective marker gene can be removed in the presence of the site-specific recombinase. In one embodiment, the differentiated somatic cells comprise a conditional knockout allele in the genome, wherein the conditional knockout allele is flanked upstream and downstream by the first and the second recombination sites such that the conditional allele can be removed in the presence of the site-specific recombinase. In one embodiment, the conditional knockout allele further comprises a selective marker gene between the first and the second recombination sites.

In one embodiment, the nucleic acid construct comprises a nucleotide sequence homologous to at least one exon of an endogenous gene being targeted, wherein the nucleotide sequence is flanked upstream and downstream by the first and the second recombination sites. In a more specific embodiment, the exon is a first exon of the endogenous gene.

In one embodiment, the nucleic acid construct comprises a nucleotide sequence homologous to at least one intron of an endogenous gene being targeted, wherein the nucleotide sequence is flanked upstream and downstream by the first and the second recombination sites.

In one embodiment, the nucleic acid construct comprises a 5′-untranslated region (UTR) upstream of an initiation codon of an endogenous gene and a 3′-untranslated region (UTR) downstream of a stop codon of the endogenous gene such that the entire endogenous gene can be replaced with the nucleic acid construct via homologous recombination.

In one embodiment, the nucleic acid construct further comprises a modified sequence of an endogenous gene being targeted, wherein the modified sequence is located outside of the region flanked by the first and the second recombination sites. In one embodiment, the modified sequence is a knock-in allele of at least one exon of the endogenous gene. In one embodiment, the modified sequence is a knock-in allele of the entire endogenous gene (i.e., “gene-swap knock-in”). The knock-in allele can be an allele that confers desirable characteristics on an animal that contains the allele, such as improved disease resistance or larger size (e.g., larger muscle size). In one embodiment, the nucleic acid construct further comprises a transgene sequence, wherein the transgene sequence is located outside of the region flanked by the first and the second recombination sites. In one embodiment, the transgene sequence encodes a human protein (e.g., insulin, alpha-lactalbumin, transferrin, human serum albumin, human growth hormone, a blood clotting factor, etc.). In one embodiment, the transgene sequence encodes a therapeutic agent (e.g., a therapeutic antibody).

In one embodiment, the nucleic acid construct further comprises a modified sequence of the endogenous gene being targeted, wherein the modified sequence is a knockout allele of an endogenous gene. In one embodiment, the knockout allele comprises a reporter gene, wherein 5′ of the reporter gene comprises a nucleotide sequence immediately upstream of an initiation codon (ATG) of an endogenous gene (i.e., 5′ untranslated region (5′-UTR)) such that transcription of the reporter gene can be initiated by an endogenous promoter that drives expression of the endogenous gene, and transcription of the endogenous gene can be abolished.

In one embodiment, the reporter gene is located upstream of the first recombination site.

In one embodiment, the reporter gene encodes a reporter protein selected from the group consisting of alkaline phosphatase, luciferase, beta-galactosidase, beta-glucuronidase, green fluorescent protein (GFP), enhanced green fluorescent protein (EGFP), cyan fluorescent protein (CFP), yellow fluorescent protein (YFP), DsRed, and ZsGreen.

In one embodiment, the self-excisable, recombinase expression cassette is located in a transcriptionally active locus in the genome of the differentiated somatic cells. In one embodiment, the transcriptionally active locus is a ROSA26 locus. In one embodiment, the transcriptionally-active locus is CH25h locus.

In one embodiment, the site-specific recombinase is selected from the group consisting of Cre, Flp, and Dre recombinases.

In one embodiment, the site-specific recombinase is a Cre recombinase.

In one embodiment, the Cre recombinase comprises an intron sequence. In one embodiment, the Cre recombinase comprises a nuclear localization signal (NLS). In one embodiment, the Cre recombinase comprises both an intron sequence and a nuclear localization signal (NLS).

In one embodiment, the first and second recombination sites are selected from the group consisting of loxP, lox511, lox2272, lox66, lox71, loxM2, lox5171, FRT, FRT11, FRT71, attp, att, FRT, and Dre sites.

In one embodiment, the ES cell-specific promoter is not active in the cloned oocyte. In one embodiment, the ES cell-specific promoter is coupled to a ligand-inducible promoter, e.g., tetracycline (tet) on/off system, in such a way that the activity of the ES cell-specific promoter is turned off in the absence of a ligand, but the promoter activity is turned on following administration of a suitable ligand, e.g., tetracycline, and in the presence of an ES cell-specific transcription factor.

In one aspect, a method for preparing a genetically modified pig or cow in an F0 generation that lacks a selection gene, comprising genetically modifying a somatic cell of a pig or cow to include a self-excising cassette comprising a site-specific recombinase gene driven by a promoter that is active in a pluripotent cell, and a selection gene flanked by recombinase sites to form a genetically modified pig or cow genome; and introducing the genetically modified genome into a suitable oocyte, culturing the oocyte to a blastocyst stage, gestating the blastocyst in a suitable surrogate mother, and allowing the blastocyst to develop into a genetically modified progeny.

In one aspect, a method for modifying a genome of a differentiated somatic cell of a cow or pig is provided, comprising: (a) introducing into a differentiated somatic cell of a cow or pig a composition comprising: (i) a first nucleic acid construct comprising a self-excisable, recombinase expression cassette containing a site-specific recombinase gene operably linked to an ES cell-specific promoter; and (ii) a second nucleic acid construct comprising a gene encoding an ES cell-specific transcription factor,

wherein the recombinase expression cassette is flanked by recombination sites that are oriented in the same direction with respect to each other such that the site-specific recombinase can be excised in the presence of the site-specific recombinase, and

wherein the ES cell-specific transcription factor is capable of activating the ES cell-specific promoter.

In one embodiment, the ES cell-specific transcription factor is at least one selected from the group consisting of Oct-3/4, Sox2, c-Myc, Kif4, Nanog, and Lin28. In one embodiment, the at least one ES cell-specific transcription factor is capable of reprogramming the differentiated somatic cell into a pluripotent stem cell.

In one embodiment, the nucleic acid construct is a targeting construct. In one embodiment, the targeting construct comprises a knockout allele. In one embodiment, the targeting construct comprises a knock-in allele. In one embodiment, the nucleic acid construct comprises a transgene.

### BRIEF DESCRIPTION OF THE DRAWINGS

FIG. 1 illustrates steps for creating a genetically engineered and cloned non-human animal.

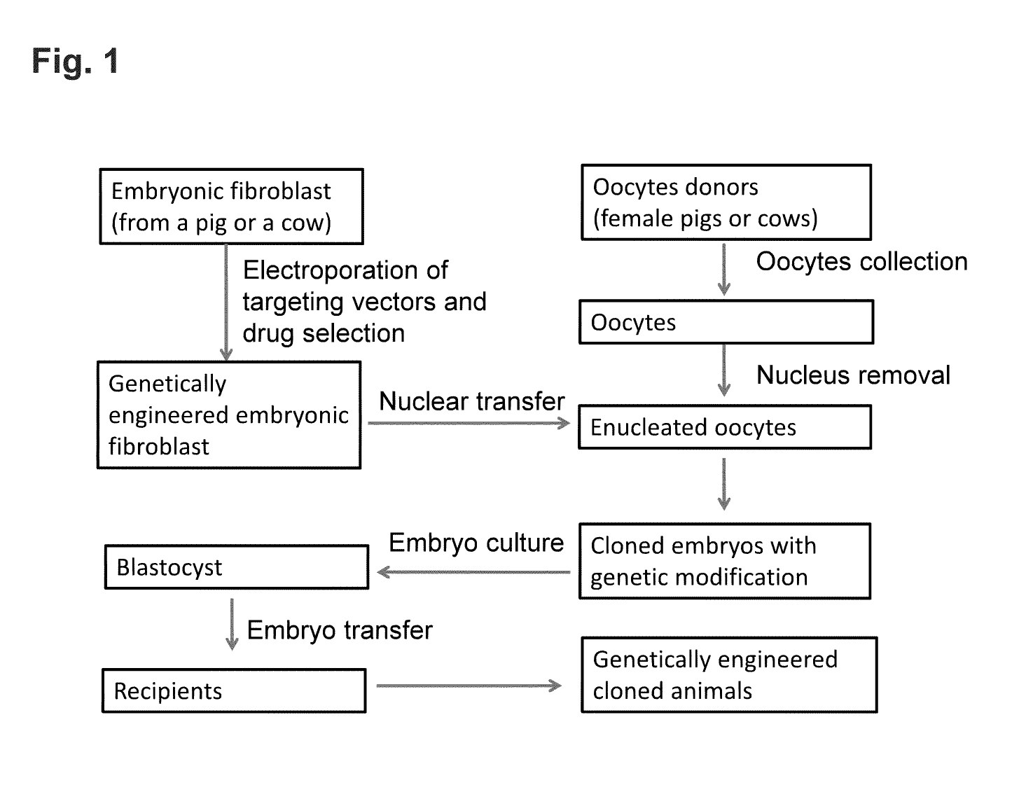

FIG. 2 illustrates a self-excisable cassette for generating a marker-free, genetically modified non-human animal.

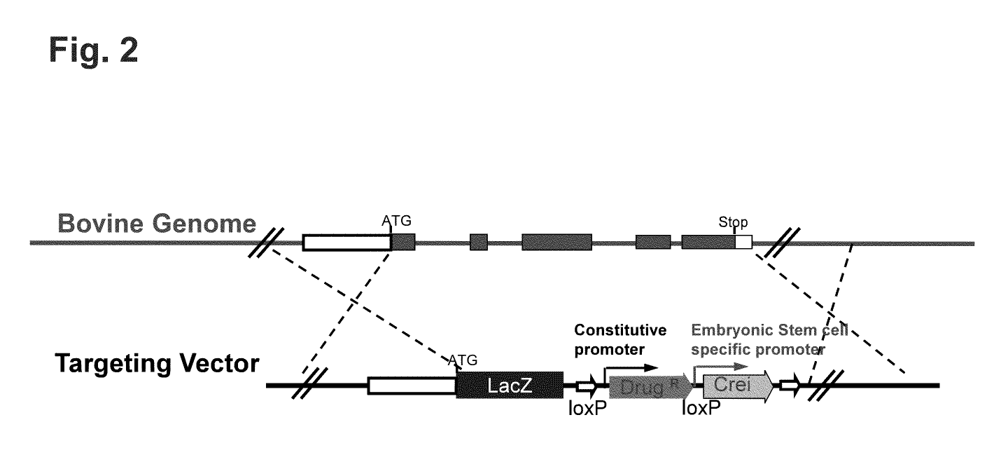

FIG. 3 illustrates steps for creating a genetically engineered and cloned non-human animal free of a selective marker.

FIG. 4 illustrates a platform for creating a genetically modified and cloned non-human animal.

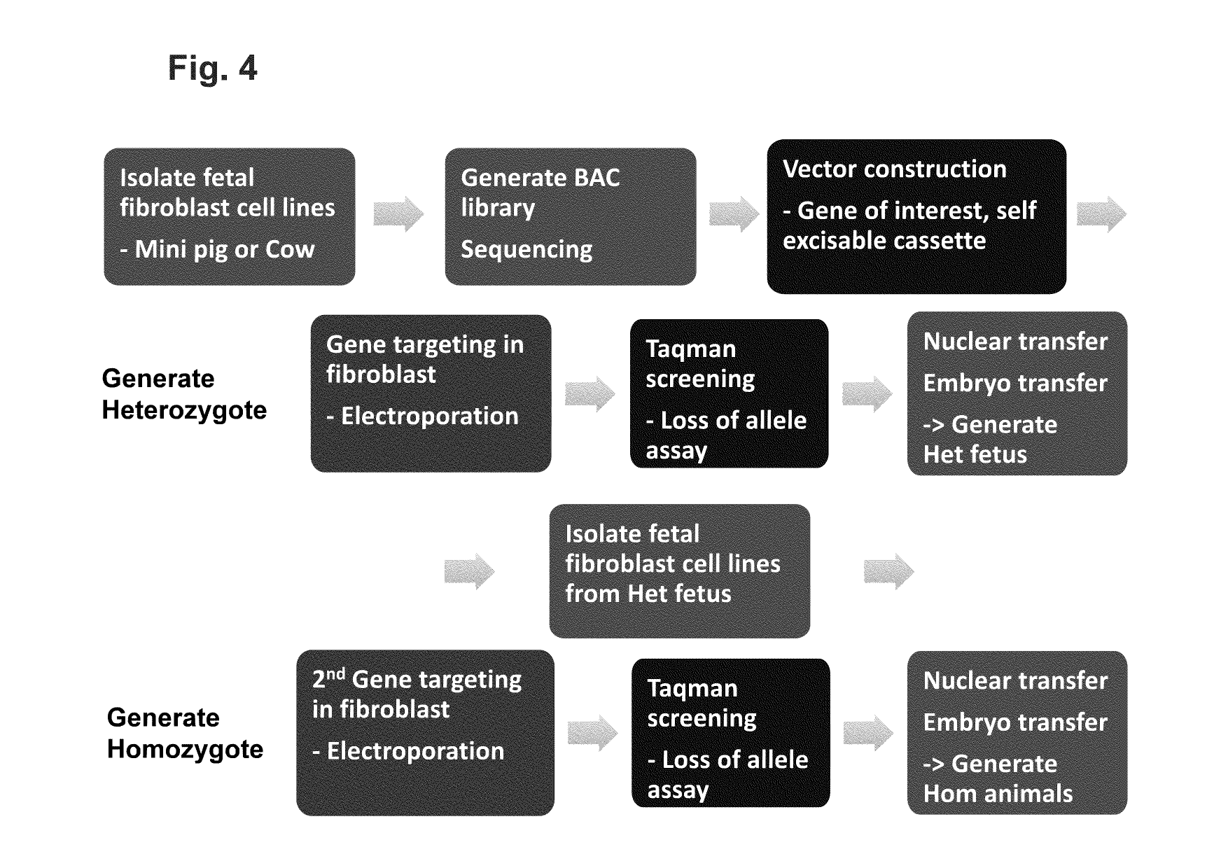

### DETAILED DESCRIPTION OF THE INVENTION

Glossary

The term “cloning” as used herein includes the process of creating an identical copy of an original organism.

The term “embryonic stem cell” or “ES cell” as used herein includes stem cells derived from the undifferentiated inner mass cells of an embryo, which, upon introduction into an embryo, can contribute to any tissue of the developing embryo.

The phrase “operably linked” as used herein includes connecting a nucleotide sequence encoding a promoter to another nucleotide sequence encoding a protein in such a way that the promoter controls expression of the nucleotide sequence encoding the protein.

The term “promoter” and “promoter regulatory element”, and the like, as used herein include a nucleotide sequence element within a nucleic acid fragment or gene that controls the expression of that gene. These can also include expression control sequences. Promoter regulatory elements, and the like, from a variety of sources can be used efficiently to promote gene expression. Promoter regulatory elements are meant to include constitutive, tissue-specific, developmental-specific, inducible, sub genomic promoters, and the like. Promoter regulatory elements may also include certain enhancer elements or silencing elements that improve or regulate transcriptional efficiency.

The term “constitutive promoter” and “constitutively active promoter” as used herein include a regulatory sequence that directs transcription of a gene in most cells or tissues at most times.

The term “pluripotent stem cell” or “multipotent stem cell” as used herein includes an undifferentiated cell that possesses the ability to develop into more than one differentiated cell types.

The term “recombination site” as used herein includes a nucleotide sequence that is recognized by a site-specific recombinase and that can serve as a substrate for a recombination event.

The term “site-specific recombinase” as used herein includes a group of enzymes that can facilitate recombination between “recombination sites” where the two recombination sites are physically separated within a single nucleic acid molecule or on separate nucleic acid molecules. Examples of “site-specific recombinase” include, but are not limited to, Cre, Flp, and Dre recombinases.

The term “somatic cell” as used herein includes any cell constituting a body of an organism that has two sets of chromosomes (2n), excluding a germ cell that has a single set of chromosome (n).

The term “somatic cell nuclear transfer” or “SCNT” as used herein includes a technique in which the nucleus of a somatic (body) cell from a donor animal, such as sheep, cattle, pigs, goats, rabbits, rats or mice, is transferred to the cytoplasm of an enucleated egg (an egg that has had its own nucleus removed). The nucleus can be subject to genetic modification by the present methods. Once inside the egg, the somatic nucleus is reprogrammed by egg cytoplasmic factors to become a zygote (fertilized egg) nucleus. The fertilized egg can then develop in vitro, e.g., to the blastocyst stage, before being transferred to a recipient animal, typically of the same species as the donor, which gives birth to an offspring containing cells clonally derived from the fertilized egg and having any genetic modification introduced into the transferred nucleus. Many somatic cell types, including mammary epithelial cells, ovarian cumulus cells, fibroblast cells from skin and internal organs, various internal organ cells, Sertoli cells, macrophage and blood leukocytes can be used (see, e.g., Tian et al., Reproductive Biology and Endocrinologyl, 1-7 (2003).

Somatic Cells Comprising a Self-Excisable, Recombinase Expression Cassette

Many efforts have been made to remove selective marker and recombinase genes from host cells or host animals following genetic modifications. For example, in order to remove a selective marker gene flanked by recombination sites, e.g., loxP or FRT, a site-specific recombinase gene is introduced into an ES cell or a fertilized egg, via, e.g., microinjection, transfection, or transduction via viral particles. Alternatively, an animal carrying a selection cassette is bred to a deleter strain that expresses a site-specific recombinase to accomplish the same effect. These techniques, however, have a number of drawbacks, including a low level of transfection efficiency in ES cells; a decrease in ES cell pluripotency due to extended in vitro culture; and additional human and financial resources required for extra breeding steps.

The present invention offers a new approach to remove selective marker and recombinase genes from a non-human animal following a genetic modification by introducing into differentiated somatic cells a self-excisable, site-specific recombinase gene driven by an ES cell-specific promoter, followed by transferring the genetically modified genome of the differentiated somatic cells into an enucleated host oocyte via, e.g., a somatic cell nuclear transfer (SCNT) technique. Upon fusion and activation, the artificially created zygote comprising the genetically modified genome of the somatic cells is cultured in vitro until it reaches a blastocyst stage and implanted into a surrogate mother for full development (See, for example, Gong et al., Generation of cloned calves from different types of somatic cells, Sci China C Life Sci, 2004, 47:470-476; incorporated herein by reference in its entirety). During development of the artificially created zygote, the site-specific recombinase becomes expressed and active in pluripotent stem cells, where ES cell-specific transcription factors are active, and the selective marker and the recombinase genes become deleted from the genome of the cloned embryo. In this way, the method obviates the need for manipulation of ES cells or any extra breeding steps required for removing selective marker and recombinase genes.

Differentiated somatic cells of non-human animal are provided, which are genetically engineered to contain a self-excisable, recombinase expression cassette comprising a site-specific recombinase gene operably linked to an ES cell-specific promoter, wherein the site-specific recombinase gene is expressed in undifferentiated pluripotent stem cells, for example, in ES cells in the inner cell mass of a blastocyst-stage embryo, but not in differentiated somatic cells.

In one aspect, differentiated somatic cells of a non-human animal that are engineered to contain a self-excisable, recombinase expression cassette are provided, wherein the self-excisable, recombinase expression cassette comprises a site-specific recombinase gene operably linked to an ES cell-specific promoter, wherein the site-specific recombinase gene is flanked upstream and downstream by a first and a second recombination sites that are oriented in the same direction with respect to each other such that the recombinase can be excised in the presence of the site-specific recombinase, and wherein the ES cell-specific promoter drives transcription of the site-specific recombinase gene in undifferentiated pluripotent stem cells but not in the somatic cells.

Once the modified genome of the differentiated somatic cells is transferred into an enucleated host oocyte, and once the artificially created zygote is allowed to develop into an embryo, the selective marker and the recombinase genes can be removed from the genome of the developing cloned embryo.

In one embodiment, the differentiated somatic cells include, but are not limited to, skin cells, blood cells, nerve cells, muscle cells, bone cells, kidney cells, liver cells, and fat cells.

In one embodiment, the differentiated somatic cells are fibroblasts. The fibroblasts can be derived from any non-human animals, including, but not limited to, a mouse, a rat, a rabbit, a bird, a cow, a pig, a sheep, a goat, a horse, and a donkey. In one embodiment, the fibroblasts are derived from a pig. In a more specific embodiment, the pig is a mini pig. In one embodiment, the fibroblasts are derived from a cow.

In one embodiment, the ES cell-specific promoter is selected from the group consisting of Oct-3/4 promoter, Sox2 promoter, Kif4 promoter, c-Myc promoter, Nanog promoter, Lin28 promoter, and a combination thereof.

In one embodiment, the ES cell-specific promoter drives transcription of the site-specific recombinase gene in ES cells of a blastocyst-stage embryo.

In one embodiment, the self-excisable, recombinase expression cassette comprises a second expression cassette between the first and the second recombination sites, wherein the second expression cassette comprises a selective marker gene operably linked to a promoter. In one embodiment, the selective marker is located upstream of the site-specific recombinase gene. In another embodiment, the selective marker is located downstream of the site-specific recombinase gene.

In one embodiment, the promoter operably linked to the selective marker gene is a constitutive promoter. In one embodiment, the constitutive promoter is selected from the group consisting of a Ubc promoter, an hCMV promoter, an mCMV promoter, an EF-1 promoter, a Pgk1 promoter, a beta-actin promoter, and a ROSA26 promoter.

In one embodiment, the selective marker is selected from the group consisting of neomycin phosphotransferase (neo^{r}), hygromycin B phosphotransferase (hyg^{r}), puromycin-Nacetyltransferase (puro^{r}), blasticidin S deaminase (bsr^{r}), xanthine/guanine phosphoribosyl transferase (gpt), and herpes simplex virus thymidine kinase (HSV-k).

In one embodiment, the self-excisable, recombinase expression construct does not comprise a selective marker gene, and the selective marker gene is located in another locus (e.g., in trans) in the genome of the somatic cell, wherein the selective marker gene is flanked upstream and downstream by third and fourth recombination sites oriented in the same direction with respect to each other such that the selective marker can be removed in the presence of the site-specific recombinase.

In one embodiment, the differentiated somatic cells comprise a conditional knockout allele in the genome, wherein the conditional knockout allele is flanked upstream and downstream by the first and the second recombination sites in such a way that the conditional allele can be removed in the presence of the site-specific recombinase. In one embodiment, the conditional knockout allele comprises a selective marker gene between the first and the second recombination sites.

In one embodiment, the self-excisable, recombinase expression construct comprises a nucleotide sequence homologous to at least one exon of an endogenous gene being targeted, wherein the nucleotide sequence is flanked upstream and downstream by the first and the second recombination sites. In a more specific embodiment, the exon is a first exon of the endogenous gene.

In one embodiment, the self-excisable, recombinase expression construct comprises a nucleotide sequence homologous to at least one intron of an endogenous gene being targeted, wherein the nucleotide sequence is flanked upstream and downstream by the first and the second recombination sites.

In one embodiment, the self-excisable, recombinase expression construct comprises a 5′-untranslated region (UTR) upstream of an initiation codon of an endogenous gene and a 3′-untranslated region (UTR) downstream of a stop codon of the endogenous gene such that the entire endogenous gene can be replaced with the targeting construct via homologous recombination.

In one embodiment, the self-excisable, recombinase expression construct further comprises a modified sequence of an endogenous gene being targeted, wherein the modified sequence is located outside of the region flanked by the first and the second recombination sites. In one embodiment, the modified sequence is a knock-in allele of at least one exon of an endogenous gene. In one embodiment, the modified sequence is a knock-in allele of the entire endogenous gene (i.e., “gene-swap knock-in”). The knock-in allele can be an allele that confers desirable characteristics on an animal that contains the allele, such as improved disease resistance or larger size (e.g., larger muscle size). In one embodiment, the nucleic acid construct further comprises a transgene sequence, wherein the transgene sequence is located outside of the region flanked by the first and the second recombination sites. In one embodiment, the transgene sequence encodes a human protein (e.g., insulin, alpha-lactalbumin, transferrin, human serum albumin, human growth hormone, a blood clotting factor, etc.). In one embodiment, the transgene sequence encodes a therapeutic agent (e.g., a therapeutic antibody).

In one embodiment, the nucleic acid construct further comprises a modified sequence of the endogenous gene being targeted, wherein the modified sequence is a knockout allele of an endogenous gene. In one embodiment, the knockout allele comprises a reporter gene, wherein 5′ of the reporter gene comprises a nucleotide sequence immediately upstream of an initiation codon (ATG) of the endogenous gene (i.e., 5′ untranslated region (5′-UTR)) such that transcription of the reporter gene can be initiated by an endogenous promoter that drives expression of the endogenous gene, and transcription of the endogenous gene can be abolished.

In one embodiment, the reporter gene is located upstream of the first recombination site.

In one embodiment, the reporter gene encodes a reporter protein selected from the group consisting of alkaline phosphatase, luciferase, beta-galactosidase, beta-glucuronidase, green fluorescent protein (GFP), enhanced green fluorescent protein (EGFP), cyan fluorescent protein (CFP), yellow fluorescent protein (YFP), DsRed, and ZsGreen.

In one embodiment, the self-excisable, recombinase expression cassette is located in a transcriptionally active locus in the genome of the differentiated somatic cells. In one embodiment, the transcriptionally active locus is a ROSA26 locus. In one embodiment, the transcriptionally-active locus is CH25h locus.

In one embodiment, the site-specific recombinase is selected from the group consisting of Cre, Flp, and Dre recombinases.

In one embodiment, the site-specific recombinase is a Cre recombinase.

In one embodiment, the Cre recombinase comprises an intron sequence. In one embodiment, the Cre recombinase comprises a nuclear localization signal (NLS). In one embodiment, the Cre recombinase comprises both an intron sequence and a nuclear localization signal (NLS).

In one embodiment, the first and second recombination sites are selected from the group consisting of loxP, lox511, lox2272, lox66, lox71, loxM2, lox5171, FRT, FRT11, FRT71, attp, att, FRT, and Dre sites.

Production of a Genetically Modified and Cloned Animals

The present invention employs a strategy to genetically modify differentiated somatic cells, e.g., fibroblasts, of a non-human animal, to harbor a self-excisable, recombinase expression cassette driven by an ES cell-specific promoter at a specific locus. Instead of manipulating ES cells in vitro for selection cassette removal, the nucleus of the genetically modified somatic cell is transferred into an enucleated host oocyte to induce reprogramming of the genome and deletion of the selection cassette in pluripotent stem cells during development of the cloned embryo. In this way, a selective marker-free, non-human animal, which is cloned from a genetically modified somatic cell, can be produced without the need for manipulating ES cells or for breeding a selection cassette-containing animal to a deleter strain that expresses a site-specific recombinase.

The method of the present invention can be employed in producing any genetically modified and cloned non-human animals. Non-limiting examples of the non-human animals include rodents (e.g., mice, rats), rabbits, birds (e.g., chickens, turkeys, ducks, geese, etc.), cows, pigs, sheep, goats, horses, and donkeys. In a preferred embodiment, the non-human animal is either a pig or a cow.

In one aspect, a method for producing a genetically modified and cloned non-human animal that is free of a selective marker gene and a recombinase gene is provided, wherein the method comprises:

(a) introducing a targeting construct into differentiated somatic cells of a non-human animal to create a genetically modified genome;

(b) transferring the genetically-modified genome of (a) into an enucleated host oocyte;

(c) fusing and activating the oocyte of (b) to form an artificial zygote;

(d) culturing the artificial zygote of (c) in vitro until the zygote reaches a blastocyst embryonic stage; and

(e) implanting the blastocyst of (d) into a uterus of a surrogate mother to form the genetically modified and cloned non-human animal that is free of the selective marker gene and the recombinase gene,

wherein the targeting construct comprises a self-excisable, recombinase expression cassette comprising a site-specific recombinase gene operably linked to an ES cell-specific promoter, wherein the recombinase expression construct is flanked upstream and downstream by a first and second recombination sites oriented in the same direction in such a way that the site-specific recombinase can be excised in the presence of the site-specific recombinase, and wherein the ES cell-specific promoter drives transcription of the site-specific recombinase gene in undifferentiated pluripotent stem cells in a developing cloned embryo but not in the differentiated somatic cells.

In one embodiment, the undifferentiated pluripotent stem cells are ES cells in inner cell mass (ICM) of a blastocyst-stage embryo.

In one embodiment, the ES cell-specific promoter is selected from the group consisting of Oct-3/4 promoter, Sox2 promoter, Kif4 promoter, c-Myc promoter, Nanog promoter, and Lin28 promoter.

Various gene transfer techniques can be employed to introduce the self-excisable, recombination expression cassette into the differentiated somatic cells, including, but not limited to, chemically-based transfection (e.g., calcium phosphate, cationic lipids such as lipofectin or lipofectamine, and cationic polymers such as DEAE-dextran or dendrimers), physical transfection techniques (e.g., microinjection, biolistic particle delivery such as a gene gun, lipid-based transfection, electroporation, sonoporation, magnetic nanoparticls, and laser-irradiation), and transduction via biological agents such as viral particles carrying the self-excisable, recombinase expression cassette. Electroporation is widely used to achieve gene transfer, particularly when the targeting construct is large in size. Gene transfer techniques are described, including, e.g., in Kim & Eberwine (2010), Anal. Bioanal. Chem. 397:3173-78, incorporated by reference. In some embodiments, the viral particles are derived from a virus selected from the group consisting of adenovirus, adeno-associated virus, SV-40, Epstein-Barr virus, retrovirus, lentivirus, baculovirus, coronavirus, herpes simplex virus, poliovirus, Semliki Forest virus, Sindbis virus, and Vaccina virus.

The nucleus containing the genetically modified genome of the differentiated somatic cells can be transferred into an enucleated host oocyte using any method known in the art (See, for example, Gong et al., Generation of cloned calves from different types of somatic cells, Sci China C Life Sci, 2004, 47:470-476; incorporated herein by reference in its entirety). In one embodiment, the genetically-modified genome of the somatic cell is transferred into the enucleated host oocyte via somatic cell nuclear transfer technique (SCNT).

In various embodiments, the self-excisable, recombinase expression cassette comprises a second expression cassette located between the first and the second recombination sites, wherein the second expression cassette comprises a selective marker gene operably linked to a promoter. In one embodiment, the selective marker is located upstream of the site-specific recombinase gene. In another embodiment, the selective marker is located downstream of the site-specific recombinase gene.

In one embodiment, the promoter operably linked to the selective marker gene is a constitutive promoter. In one embodiment, the constitutive promoter is selected from the group consisting of a Ubc promoter, an hCMV promoter, an mCMV promoter, an EF-1 promoter, a Pgk1 promoter, a beta-actin promoter, and a ROSA26 promoter.

In one embodiment, the selective marker is selected from the group consisting of neomycin phosphotransferase (neo^{r}), hygromycin B phosphotransferase (hyg^{r}), puromycin-Nacetyltransferase (puro^{r}), blasticidin S deaminase (bsr^{r}), xanthine/guanine phosphoribosyl transferase (gpt), and herpes simplex virus thymidine kinase (HSV-k).

In one embodiment, the self-excisable, recombinase expression construct does not comprise a selective marker gene, and the selective marker gene is located in another locus (e.g., in trans) in the genome of the differentiated somatic cell, wherein the selective marker gene is flanked upstream and downstream by third and fourth recombination sites oriented in the same direction with respect to each other such that the selective marker can be removed in the presence of the site-specific recombinase. In one embodiment, the differentiated somatic cells comprise a conditional knockout or knock-in allele in the genome, wherein the conditional knockout or knock-in allele is flanked upstream and downstream by the first and the second recombination sites such that the conditional knockout or knock-in allele can be removed from the genome in the presence of the site-specific recombinase. In one embodiment, the conditional knockout or knock-in allele comprises a selective marker gene between the first and the second recombination sites.

In one embodiment, the targeting construct comprises a nucleotide sequence homologous to at least one exon of an endogenous gene being targeted, wherein the nucleotide sequence is flanked upstream and downstream by the first and the second recombination sites. In one embodiment, the exon is a first exon of the endogenous gene.

In one embodiment, the targeting construct comprises a nucleotide sequence homologous to at least one intron of an endogenous gene being targeted, wherein the nucleotide sequence is flanked upstream and downstream by the first and the second recombination sites.

In one embodiment, targeting arms of the targeting construct comprise a 5′-untranslated region (UTR) upstream of an initiation codon of an endogenous gene and a 3′-untranslated region (UTR) downstream of a stop codon of the endogenous gene such that the entire endogenous gene can be replaced with the targeting construct via homologous recombination. In one embodiment, the targeting arms comprise a 5′-UTR immediately upstream of an initiation codon. In one embodiment, the targeting arms comprise a 3′-UTR immediately downstream of a stop codon of the endogenous gene.

In one embodiment, the targeting construct further comprises a modified sequence of an endogenous gene being targeted, wherein the modified sequence is located outside of the region flanked by the first and the second recombination sites. In one embodiment, the modified sequence is a knock-in allele of at least one exon of the endogenous gene. In one embodiment, the modified sequence is a knock-in allele of the entire endogenous gene (i.e., “gene-swap knock-in”). The knock-in allele can be an allele that confers desirable characteristics on an animal that contains the allele, such as improved disease resistance or larger size (e.g., larger muscle size).

In one embodiment, the targeting construct further comprises a transgene sequence, wherein the transgene sequence is located outside of the region flanked by the first and the second recombination sites. In one embodiment, the transgene sequence encodes a human protein (e.g., insulin, alpha-lactalbumin, transferrin, human serum albumin, human growth hormone, a blood clotting factor, etc.). In one embodiment, the transgene sequence encodes a therapeutic agent (e.g., a therapeutic antibody).

In one embodiment, the targeting construct further comprises a modified sequence of an endogenous gene being targeted, wherein the modified sequence is a knockout allele of an endogenous gene. In one embodiment, the knockout allele comprises a reporter gene, wherein 5′ of the reporter gene comprises a nucleotide sequence immediately upstream of an initiation codon (ATG) of the endogenous gene (i.e., 5′ untranslated region (5′-UTR)) such that transcription of the reporter gene can be initiated by an endogenous promoter that drives the endogenous gene, and transcription of the endogenous gene can be abolished.

In one embodiment, the reporter gene is located upstream of the first recombination site. In one embodiment, the reporter gene encodes a protein selected from the group consisting of green fluorescent protein (GFP), enhanced green fluorescent protein (EGFP), cyan fluorescent protein (CFP), yellow fluorescent protein (YFP), DsRed, ZsGreen, and lacZ.

In one embodiment, the genetically modified genome of the differentiated somatic cells is microinjected into a perivitelline space (i.e., the space between the zona pellucida and the cell membrane) of the host enucleated oocyte.

In one embodiment, the expression construct comprises a selective marker gene operably linked to a promoter. In one embodiment, the promoter is a constitutive promoter. In one embodiment, the constitutive promoter is selected from the group consisting of a Ubc promoter, an hCMV promoter, an mCMV promoter, an EF-1 promoter, a Pgk1 promoter, a beta-actin promoter, and a ROSA26 promoter.

In one embodiment, the selective marker gene is located upstream of the site-specific recombinase gene. In one embodiment, the selective marker gene is located downstream of the site-specific recombinase.

In one embodiment, the selective marker is a drug resistant gene selected from the group consisting of neomycin phosphotransferase (neo^{r}), hygromycin B phosphotransferase (hyg^{r}), puromycin-Nacetyltransferase (puro^{r}), blasticidin S deaminase (bsr^{r}), xanthine/guanine phosphoribosyl transferase (gpt), and herpes simplex virus thymidine kinase (HSV-k).

In one embodiment, the site-specific recombinase is selected from the group consisting of Cre, Flp, and Dre recombinases.

In one embodiment, the site-specific recombinase is a Cre recombinase. In one embodiment, the Cre recombinase comprises an intron sequence. In one embodiment, the Cre recombinase comprises a nuclear localization signal (NLS). In one embodiment, the Cre recombinase comprises both an intron sequence and a nuclear localization signal (NLS).

In one embodiment, the first and second recombination sites are selected from the group consisting of loxP, lox511, lox2272, lox66, lox71, loxM2, lox5171, FRT, FRT11, FRT71, attp, att, FRT, and Dre sites

In one aspect, a method for producing genetically modified and cloned pluripotent stem cells of a non-human animal that are free of a selective marker gene and a recombinase gene is provided, comprising:

(a) introducing a targeting construct into differentiated somatic cells of a non-human animal to create a genetically modified genome; and

(b) transferring the genetically-modified genome of (a) into pluripotent stem cells to produce the genetically modified and cloned pluripotent stem cells free of the selective marker gene and the recombinase gene,

wherein the targeting construct comprises a self-excisable, recombinase expression cassette comprising a site-specific recombinase gene operably linked to an ES cell-specific promoter, wherein the recombinase expression construct is flanked upstream and downstream by a first and second recombination sites that are oriented in the same direction such that the site-specific recombinase can be excised in the presence of the site-specific recombinase, and wherein the ES cell-specific promoter drives transcription of the site-specific recombinase gene in the cloned pluripotent stem cells but not in the differentiated somatic cells.

Thus, the selective marker and the recombinase genes can be removed from the genome of the cloned pluripotent stem cells following transfer of the genetically modified genome of the differentiated somatic cells into pluripotent stem cells or any somatic cells reprogrammed to be pluripotent, where ES cell-specific transcription factors are active.

In one embodiment, the ES cell-specific promoter is selected from the group consisting of Oct-3/4 promoter, Sox2 promoter, Kif4 promoter, c-Myc promoter, Nanog promoter, and Lin28 promoter.

In one embodiment, the pluripotent stem cells are ES cells of a non-human animal.

In one embodiment, the pluripotent stem cells are induced pluripotent stem cells (iPS cells).

In one embodiment, the transferring step (b) is carried out via a somatic cell nuclear transfer (SCNT) technique.

Unless defined otherwise, all technical and scientific terms used herein have the same meaning as commonly understood by one of ordinary skill in the art to which this invention belongs. Although any methods and materials similar or equivalent to those described herein also can be used in the practice or testing of the described invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates, which may need to be independently confirmed.

The described invention may be embodied in other specific forms without departing from the spirit or essential attributes thereof and, accordingly, reference should be made to the appended claims, rather than to the foregoing specification, as indicating the scope of the invention

### EXAMPLES

The following examples are provided to describe to those of ordinary skill in the art how to make and use methods and compositions of the invention, and are not intended to limit the scope of what the inventors regard as their invention. Efforts have been made to ensure accuracy with respect to numbers used (e.g., amounts, temperatures, and the like) but some experimental errors and deviations should be accounted for. Unless indicated otherwise, parts are parts by weight, molecular weight is average molecular weight, temperature is in degrees Centigrade, and pressure is at or near atmospheric.

### Example 1

### Production of Heterozygous Genetically Modified Animals Free of a Selection Marker

Genetic modification is carried out in fetal fibroblasts isolated from a pig, preferably a mini pig. Using genomic DNA isolated from the pig fetal fibroblasts, a bacterial artificial chromosome (BAC) library is created, and a targeting vector containing gene of interest or portions thereof (“a targeted allele”) is designed and constructed.

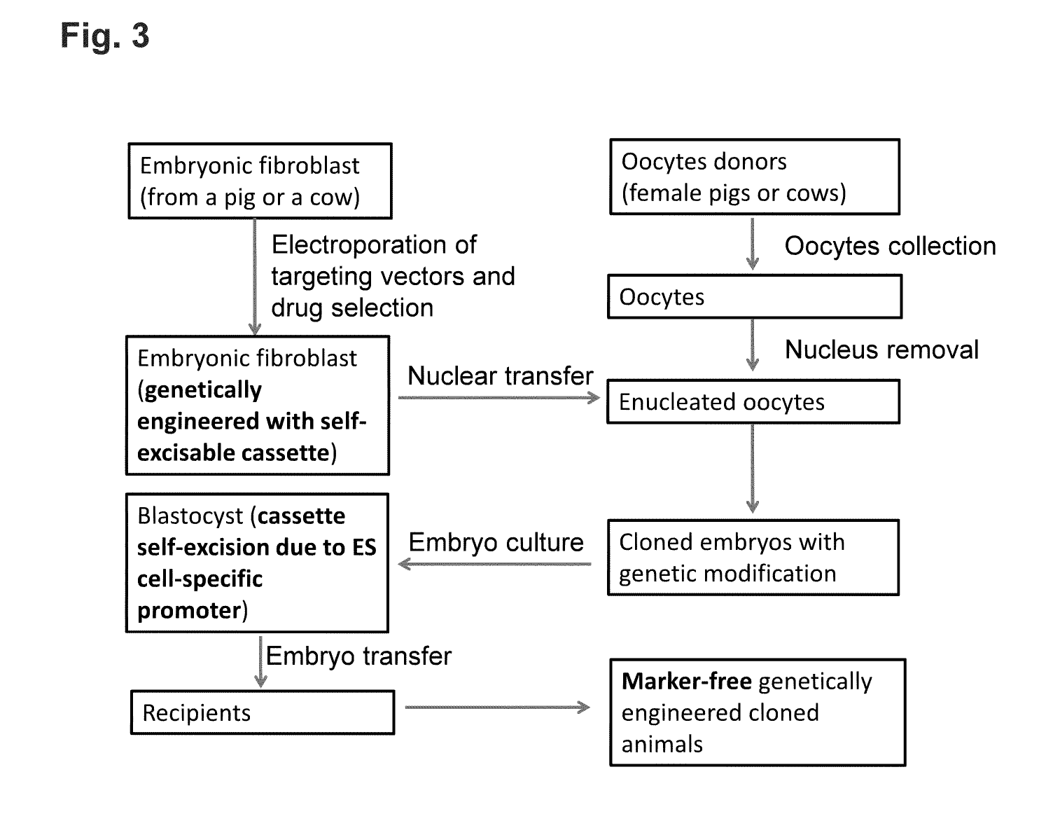

In this example, the targeting vector is designed to replace all or a portion of the coding region of an endogenous target gene with a reporter gene. The targeting vector is designed to contain a self-excisable recombinase expression cassette in which both (i) a neomycin resistant gene, which is operably linked to a constitutive promoter (e.g., ubiquitin promoter) and (ii) a Cre recombinase gene (Crei), which is operably linked to an ES cell-specific promoter (e.g., Nanog promoter) are flanked 5′ and 3′ by loxP recombination sites. In addition, at the 5′ upstream of the floxed recombinase expression cassette, the targeting vector contains the lacZ gene operably linked to a nucleotide sequence immediately upstream of an initiation codon (ATG) of an endogenous gene being targeted (i.e., 5′ untranslated region (5′-UTR)) such that, following successful gene targeting, transcription of the reporter gene (lacZ) can be initiated by an endogenous promoter that drives expression of the endogenous gene, and transcription of the endogenous gene can be abolished (See, for example, FIG. 3). The 3′ end of the targeting vector includes the 3′ untranslated region (3′-UTR) of the target gene (or pig genomic DNA flanking the 3′-UTR of the target gene). Other combinations of constitutive and ES cell-specific promoters can be used in the targeting vector.

The targeting vector is then introduced into the fetal fibroblasts via electroporation or nucleofection, and the presence of the targeted allele is confirmed by analytical PCR (e.g., real-time PCR) using specific probes and primers.

Once successful genetic modification of the fetal fibroblasts is confirmed, the fibroblasts containing one copy of the targeted allele (i.e., heterozygous for the targeted allele) are transferred into an enucleated host oocytes via somatic cell nuclear transfer (SCNT) (See, for example, Gong et al., Generation of cloned calves from different types of somatic cells, Sci China C Life Sci, 2004, 47:470-476; incorporated herein by reference in its entirety). Upon fusion and activation, the cloned zygote is cultured in vitro until it reaches a blastocyst embryonic stage, and the blastocyst-stage embryo is subsequently implanted into a surrogate mother for full development into a gene-targeted animal heterozygous for the targeted allele.

During development of the cloned embryo, the neomycin resistant gene and the Cre recombinase gene are removed from the pluripotent stem cells that express ES cell-specific transcription factors. The absence of the neomycin resistant gene and the Cre recombinase gene can be confirmed via analytical PCR (e.g., real-time PCR) using specific probes and primers or via western blot or ELISA analysis.

The gender of the resulting gene-targeted heterozygous pig depends on the gender of the pig from which the electroporated pig fetal fibroblasts were isolated, with pig fetal fibroblasts isolated from female pigs giving rise to female gene-targeted pigs and pig fetal fibroblasts isolated from male pigs giving rise to male gene-targeted pigs.

This procedure can be adapted and applied to other animals, including domesticated mammals such as cows, other types of cattle, goats, sheep, rabbits, rats, or mice.

### Example 2

### Production of Homozygous Genetically Modified Animals Free of a Selection Marker

In order to produce an animal homozygous for the targeted allele, fetal fibroblasts are isolated from the animal heterozygous for the targeted allele. The targeting vector, which is used to create the heterozygous animal, is introduced into the heterozygous fetal fibroblasts via electroporation or nucleofection. The zygosity of the targeted allele is analyzed and confirmed via analytical PCR (e.g., real-time PCR) using specific probes and primers.

The fetal fibroblasts containing a genome homozygous for the targeted allele are then transferred into enucleated host oocytes. Upon fusion and activation, the cloned zygotes (which are homozygous for the target allele) are cultured in vitro until they reach the blastocyst embryonic stage. The blastocyst stage embryos are then implanted into a surrogate mother for full development into gene-targeted animals homozygous for the targeted allele.

During development of the cloned embryos, the neomycin resistant gene and the Cre recombinase gene are removed from pluripotent stem cells that express ES cell-specific transcription factors. The absence of the neomycin resistant gene and the Cre recombinase gene can be confirmed via analytical PCR (e.g., real-time PCR) using specific probes and primers or via western blot or ELISA analysis.

This procedure can, of course, be adapted and applied to other animals, including domesticated mammals such as cows, other types of cattle, goats, sheep, rabbits, rats, or mice.

### Example 3

### Production of Genetically Modified Cloned Animals Using BAC Targeting Vectors

The gene targeting steps in Examples 1 and 2 can be performed using a targeting vector that has relatively short (e.g., 4 kb-8 kb) 5′ and 3′ homology arms (i.e., the sequences flanking the self-excisable recombinase expression cassette that are homologous with regions upstream and downstream of the target insertion site, respectively). In such instances, the targeting vector is typically less than 20 kb or 25 kb in size. Alternatively, the methods can be performed with bacterial artificial chromosome (BAC)-based targeting vectors, which can be up to several hundred kb in length and tend to produce fewer random integration events and aberrant targeting events (e.g., targeting events that are accompanied by gene rearrangement and/or deletions).

The use of BAC-based targeting vectors for gene targeting has been described in Valenzuela et al. (2003), Nature Biotechnology 21(6): 652-59, the contents of which are incorporated herein by reference. Briefly, once a BAC covering the target gene has been identified, a self-excisable, recombinase expression cassette is inserted into the target gene by bacterial homologous recombination. Although not necessary, a portion of the gene target is often deleted from the BAC during the insertion of the self-excisable recombinase expression cassette.

Once the BAC has been modified so as to create the targeting vector, the targeting vector is introduced into somatic cells (e.g., fetal fibroblasts), as described in Example 1. Because of their large size, BAC targeting vectors are most commonly introduced by electroporation or nucleofection. Following selection and isolation, BAC transformants are screened to determine whether the targeting event was a success. Such screening can be performed, for example, using an amplification-based “loss of native allele” assay provided that the 5′ and 3′ homology arms of the BAC targeting vector are non-isogenic with the corresponding target gene sequences, as described in Valenzuela et al. (supra).

After properly targeted somatic cells have been generated, somatic cell nuclear transfer can be used to generate a cloned embryo that is heterozygous for the targeted allele, as described in Example 1. Furthermore, cells from the cloned embryo (e.g., fetal fibroblasts) can be retargeted to generate a cloned embryo that is homozygous for the targeted allele, as described in Example 2.

### Example 4

### Production of Genetically Modified Cloned Livestock Having Economically Favorable Traits

Animal husbandry has sought to use breeding to produce animals that combine the beneficial traits of different animal breeds. However, animal breeding has proven inadequate in a number of regards, particularly when (1) traits are closely linked, and (2) a desirable trait in one of the breeds is a complex, polygenic trait. In addition, animal breeding can only be used to combine traits that exist in animals of the same species. Genetic engineering, which does not suffer from any of these drawbacks, has therefore begun to complement traditional animal breeding techniques. However, the presence of non-native genes (e.g., selective marker genes and/or recombinase genes) in the genetically engineered animals remains a source of concern, particularly when the animals are being used to produce products for human consumption, such as food and pharmaceuticals. Accordingly, the genetically modified, cloned animals produced by the present methods help to alleviate such concerns.

Traits considered desirable in livestock maintained for human consumption can include, for example, disease resistance, overall size, or muscle mass. For many traits of interest, animal breeders have identified genes that are responsible for or contribute to the desired characteristics. As an example, myostatin is a gene that suppresses muscle growth in animals. In cattle (as well as in dogs and mice), the presence of mutations that eliminate myostatin function has been shown to increase muscle mass. See, e.g., McPherron et al. (1997), Nature 387(6628): 83-90; Kambadur et al. (1997), Genome Res. 7(9): 910-6; Grobet et al. (1997), Nat. Genet. 17(1): 71-4; Mosher et al. (2007), PLoS Genet. 3(5):e79. Meat from cattle homozygous for a loss-of-function myostatin mutation is currently sold as a specialty product. However, the increased muscular physique of the cattle requires special handling and diet, making their meat too expensive for broad marketing. Animals heterozygous for a loss-of-function myostatin mutation also have enhanced muscle mass, though to a lesser extent than the homozygous mutants. One advantage of the heterozygous animals is that they do not require the special handling and diet required by the homozygous animals.

The present invention can therefore be applied to the production of livestock having increased muscle mass. Myostatin genes in animals such as pigs, goats, sheep, rabbits, and various types of cattle can be identified and used to produce targeting constructs having a complete or partial loss-of-function myostatin allele and a self-excisable, recombinase expression cassette. The targeting constructs can be used to produce cloned animals according to, for example, the method of Example 1, which are heterozygous for a mutant myostatin allele (e.g., a loss-of-function allele). Alternatively, the targeting constructs can be used to produce cloned animals according to, for example, the method of Example 2, which are homozygous for a mutant myostatin allele (e.g., a partial loss-of-function allele). Because the animals lack selective marker and recombinase genes otherwise associated with genetic engineering, the livestock having increased muscle mass can provide a superior source of meat that avoids concerns raised by food products produced by existing genetic engineering techniques.

### Example 5

### Production of Genetically Modified Cloned Mammals Producing Engineered Milk

Milk and dairy products produced from milk, particularly milk from cows and goats, constitute a major part of the Western diet. Significant work has been done to genetically engineer such animals to produce milk having superior nutritional value. See, e.g., Magnus and Lali (2008), Veterinary World 1(10):319-20. The present methods can be applied analogously to facilitate the production of such milk from genetically engineered animals that are free of selective marker and recombinase genes.

For example, using the method of Example 1, cows (or goats or sheep) can be engineered to express human lactoferrin or human alpha-lactalbumin in their milk. Milk containing human alpha-lactalbumin is more nutritionally balanced than, e.g., natural cows' milk, and is better suited for consumption by babies and the elderly. See Magnus and Lali, supra. Human lactoferrin is beneficial because it plays a role in stimulating the immune system and acting as a first line of defense against infection. The human gene encoding either protein can be introduced into a targeting construct of the invention having a self-excisable, recombinase expression cassette. The human gene can be placed under the control of a milk-specific promoter (e.g., the promoter for the corresponding cow gene or, alternatively, a whey acidic protein promoter) and the human gene and recombinase expression cassette can be flanked by 5′ and 3′ homology arm homologous to an appropriate region in the cow's genome (e.g., the corresponding cow gene or a non-essential region that allows for proper transgene expression). The targeting construct could then be used to produce genetically modified somatic cells and heterozygous or homozygous genetically altered, cloned cows (or goats or sheep) according to the methods of Example 1 or 2.

Because of the high rate of protein production in mammary glands, among other reasons, production of pharmaceutical proteins in milk is another area of considerable ongoing research. Important pharmaceutical agents such as antibodies, insulin, human growth hormone, blood clotting factors, and human serum albumin have all been produced and secreted into in cows' milk. See Houdebine (2009), Comp. Immun. Microbiol. Infect. Dis. 32:107-121. Each of these proteins can be beneficially produced in genetically modified animals (e.g., cows, goats, pigs) produced according to the present methods, thereby avoiding regulatory and consumer concerns about the impact of selective marker and recombinase genes on the pharmaceutical products. As discussed above, the human gene can be placed under the control of a milk-specific promoter (e.g., a whey acidic protein promoter) and the human gene and recombinase expression cassette can be flanked by 5′ and 3′ homology arm homologous to an appropriate region in the target animal's genome (e.g., a non-essential region that allows for proper transgene expression). The targeting construct can then be used to produce genetically modified somatic cells and heterozygous or homozygous genetically altered, cloned animals according to the methods of Example 1 or 2.

What is claimed is:

1. A genetically modified somatic cell of a non-human animal comprising a self-excisable, recombinase expression cassette having a site-specific recombinase gene operably linked to an embryonic stem (ES) cell-specific promoter,
wherein the site-specific recombinase cassette is flanked upstream and downstream by first and second recombination sites that are oriented in the same direction with respect to each other in such a way that the cassette can be excised in the presence of the site-specific recombinase, and
wherein the ES cell-specific promoter drives transcription of the site-specific recombinase gene in undifferentiated pluripotent stem cells but not in the genetically modified somatic cells.

2. The genetically modified somatic cell of claim 1, wherein the somatic cell is selected from the group consisting of a skin cell, blood cell, nerve cell, muscle cell, bone cell, kidney cell, liver cell, and a fat cell.

3. The genetically modified somatic cell of claim 1, wherein the somatic cell is a fibroblast.

4. The genetically modified somatic cell of claim 3, wherein the fibroblast is derived from a pig.

5. The genetically modified somatic cell of claim 4, wherein the pig is a mini pig.

6. The genetically modified somatic cell of claim 3, wherein the fibroblast is derived from a cow.

7. The genetically modified somatic cell of claim 1, wherein the ES cell-specific promoter is selected from the group consisting of Oct-3/4 promoter, Sox2 promoter, Kif4 promoter, c-Myc promoter, Nanog promoter, Lin28 promoter, and a combination thereof.

8. The genetically modified somatic cell of claim 1, wherein the ES cell-specific promoter drives transcription of the site-specific recombinase gene in ES cells of a blastocyst-stage embryo.

9. The genetically modified somatic cell of claim 1, wherein the self-excisable, recombinase expression cassette further comprises a selective marker gene between the first and second recombination sites, wherein the selective marker gene is operably linked to a promoter.

10. The genetically modified somatic cell of claim 9, wherein the promoter operably linked to the selective marker gene is a constitutive promoter.

11. The genetically modified somatic cell of claim 1, wherein the self-excisable, recombinase expression cassette does not comprise a selective marker gene, and the selective marker gene is located in another locus in the genome of the somatic cell, and wherein the selective marker gene is flanked upstream and downstream by third and fourth recombination sites that are oriented in the same direction with respect to each other such that the selective marker can be removed in the presence of the site-specific recombinase.

12. The genetically modified somatic cell of claim 1, wherein the genetically modified genome of the somatic cell comprises a conditional knockout allele, and wherein the conditional knockout allele is flanked upstream and downstream by the first and second recombination sites such that the conditional allele can be removed in the presence of the site-specific recombinase.

13. The genetically modified somatic cell of claim 1, wherein the recombinase expression cassette comprises a nucleotide sequence homologous to at least one exon of an endogenous gene being targeted, and wherein the nucleotide sequence is flanked upstream and downstream by the first and second recombination sites.

14. The genetically modified somatic cell of claim 1, wherein the recombinase expression cassette comprises a nucleotide sequence homologous to at least one intron of an endogenous gene being targeted, and wherein the nucleotide sequence is flanked upstream and downstream by the first and second recombination sites.

15. The genetically modified somatic cell of claim 1, wherein the self-excisable, recombinase expression cassette is located in a transcriptionally-active locus in the genome of the somatic cell.

16. The genetically modified somatic cell of claim 1, wherein the site-specific recombinase is selected from the group consisting of Cre, Flp, and Dre recombinases.

17. A method for producing a genetically modified and cloned non-human animal that is free of a selective marker gene and a recombinase gene, the method comprising:
(a) introducing a nucleic acid construct into a differentiated somatic cell of a non-human animal to create a genetically modified genome;
(b) transferring the genetically modified genome of (a) into an enucleated host oocyte;
(c) fusing and activating the oocyte of (b) to form an artificial zygote;
(d) culturing the artificial zygote of (c) until the zygote reaches a blastocyst embryonic stage; and
(e) implanting the blastocyst of (d) into a uterus of a surrogate mother to form the genetically modified, cloned non-human animal that is free of the selective marker gene and the recombinase gene,
wherein the nucleic acid construct comprises a self-excisable, recombinase expression cassette comprising a site-specific recombinase gene operably linked to an ES cell-specific promoter,
wherein the recombinase expression cassette is flanked upstream and downstream by first and second recombination sites that are oriented in the same direction such that the cassette can be excised in the presence of the site-specific recombinase, and
wherein the ES cell-specific promoter drives transcription of the site-specific recombinase gene in undifferentiated pluripotent stem cells but not in the differentiated somatic cell.

18. The method according to claim 17, wherein the ES cell-specific promoter is selected from the group consisting of Oct-3/4 promoter, Sox2 promoter, Kif4 promoter, c-Myc promoter, Nanog promoter, Lin28 promoter, and a combination thereof.

19. The method according to claim 17, wherein the self-excisable, recombinase expression cassette comprises a selective marker gene located between the first and second recombination sites, and wherein the selective marker gene is operably linked to a promoter.

20. The method according to claim 19, wherein the promoter operably linked to the selective marker gene is a constitutive promoter.

21. The method according to claim 17, wherein the self-excisable, recombinase expression cassette does not comprise a selective marker gene, and the selective marker gene is located in another locus in the genome of the somatic cell, and wherein the selective marker gene is flanked upstream and downstream by third and fourth recombination sites that are oriented in the same direction with respect to each other such that the selective marker can be removed in the presence of the site-specific recombinase.

22. The method according to claim 17, wherein the somatic cell comprises a conditional knockout allele in the genome, and wherein the conditional knockout allele is flanked upstream and downstream by the first and second recombination sites such that the conditional allele can be removed in the presence of the site-specific recombinase.

23. The method according to claim 17, wherein the nucleic acid construct comprises a nucleotide sequence homologous to at least one exon of an endogenous gene being targeted, and wherein the nucleotide sequence is flanked upstream and downstream by the first and second recombination sites.

24. The method according to claim 17, wherein the nucleic acid construct comprises a nucleotide sequence homologous to at least one intron of an endogenous gene being targeted, and wherein the nucleotide sequence is flanked upstream and downstream by the first and second recombination sites.

25. The method according to claim 17, wherein the nucleic acid construct comprises a selective marker gene operably linked to a promoter.

26. The method according to claim 23, wherein the promoter is a constitutive promoter.

27. The method according to claim 17, wherein the site-specific recombinase is selected from the group consisting of Cre, Flp, and Dre recombinases.

28. A cloned oocyte of a non-human animal comprising a genetically modified genome from a differentiated somatic cell, wherein the genetically modified genome comprises a self-excisable, recombinase expression cassette having a site-specific recombinase gene operably linked to an ES cell-specific promoter, and wherein the self-excisable, recombinase expression cassette is flanked by first and second recombination sites oriented in the same direction with respect to each other such that the cassette can be excised in the presence of the site-specific recombinase.

29. The cloned oocyte according to claim 28, wherein the non-human animal is selected from the group consisting of a mouse, a rat, a rabbit, a bird, a cow, a pig, a sheep, a goat, a horse, and a donkey.

30. The cloned oocyte according to claim 28, wherein the differentiated somatic cell is selected from the group consisting of a skin cell, a blood cell, a nerve cell, a muscle cell, a bone cell, a liver cell, and a fat cell.

31. The cloned oocyte according to claim 28, wherein the differentiated somatic cell is a fibroblast.

32. The cloned oocyte according to claim 31, wherein the fibroblast is derived from a pig.

33. The cloned oocyte according to claim 32, wherein the pig is a mini pig.

34. The cloned oocyte according to claim 31, wherein the fibroblast is derived from a COW.

35. The cloned oocyte according to claim 28, wherein the ES cell-specific promoter is selected from the group consisting of Oct-3/4 promoter, Sox2 promoter, Kif4 promoter, c-Myc promoter, Nanog promoter, Lin28 promoter, and a combination thereof.

36. The cloned oocyte according to claim 28, wherein the ES cell-specific promoter drives transcription of the site-specific recombinase gene in ES cells of a blastocyst-stage embryo.

37. The cloned oocyte according to claim 28, wherein the self-excisable, recombinase expression cassette comprises a selective marker gene between the first and second recombination sites, and wherein the selective marker gene is operably linked to a promoter.

38. The cloned oocyte according to claim 37, wherein the promoter operably linked to the selective marker gene is a constitutive promoter.

39. The cloned oocyte according to claim 28, wherein the self-excisable, recombinase expression cassette does not comprise a selective marker gene, and the selective marker gene is located in another locus in the somatic cell genome, and wherein the selective marker gene is flanked upstream and downstream by third and fourth recombination sites that are oriented in the same direction with respect to each other such that the selective marker can be removed in the presence of the site-specific recombinase.

40. The cloned oocyte according to claim 28, wherein the somatic cells comprise a conditional knockout allele in a genome, and wherein the conditional knockout allele is flanked upstream and downstream by the first and the second recombination sites such that the conditional allele can be removed in the presence of the site-specific recombinase.

41. The cloned oocyte according to claim 28, wherein the self-excisable, recombinase expression cassette comprises a nucleotide sequence homologous to at least one exon of an endogenous gene being targeted, and wherein the nucleotide sequence is flanked upstream and downstream by the first and second recombination sites.

42. The cloned oocyte according to claim 28, wherein the self-excisable, recombinase expression cassette comprises a nucleotide sequence homologous to at least one intron of an endogenous gene being targeted, and wherein the nucleotide sequence is flanked upstream and downstream by the first and second recombination sites.

43. The cloned oocyte according to claim 28, wherein the self-excisable, recombinase expression cassette is located in a transcriptionally-active locus in the somatic cell genome.

44. The cloned oocyte according to claim 28, wherein the site-specific recombinase is selected from the group consisting of Cre, Flp, and Dre recombinases.
